(19) United States
(12) Patent Application Publication
(10) Pub. No.: US 2013/0310659 A1
(43) Pub. Date: Nov. 21, 2013

(54) BLOOD PRESSURE MEASURING DEVICE

(71) Applicant: DENSO CORPORATION, Kariya-city (JP)

(72) Inventors: Taiji KAWACHI, Kariya-shi (JP); Tadao OHOKA, Nagoya (JP)

(73) Assignee: DENSO CORPORATION, Kariya-city (JP)

(21) Appl. No.: 13/872,200

(22) Filed: Apr. 29, 2013

(30) Foreign Application Priority Data
May 1, 2012 (JP) 2012-104496

(51) Int. Cl. A61B 5/0404; A61B 5/01; A61B 5/0456; A61B 5/0205; A61B 5/0408; A61B 5/04

(52) U.S. Cl. CPC A61B 5/0404; A61B 5/04085; A61B 5/04012; A61B 5/0456; A61B 5/02055; A61B 5/01
USPC 600/301; 600/393

(57) ABSTRACT

In a blood pressure measuring device, a casing is held in both hands. A pair of electrocardiographic electrodes are respectively provided to allow contact with the hands holding the casing, and detect electrocardiographic signals through the hands. A pulse wave sensor is provided to allow contact with either of the hands holding the casing, and detects pulse wave signals through the hand. Based on these detected signals, a measuring section acquires measurement information including: a time difference between an electrocardiographic R wave and a pulse wave reference point; and a pulse wave amplitude. A calculating section calculates blood pressure using the measurement information. A display section displays the blood pressure. A measurement starting section enables the measuring section to start acquisition of the measurement information in a state in which contact is maintained between the hands holding the casing and the corresponding electrocardiographic electrodes and pulse wave sensor.

### CROSS-REFERENCE TO RELATED APPLICATION

This application is based on and claims the benefit of priority from earlier Japanese Patent Application No. 2012-104496 filed May 1, 2012, the description of which is incorporated herein by reference.

### BACKGROUND

1. Technical Field

The present invention relates to a blood pressure measuring device.

2. Related Art

As this type of measuring device, a health management index advising apparatus is known that includes electrodes for applying current and electrodes for measuring voltage in correspondence with both hands (for example, refer to JP-A-H10-174680). Specifically, the health management index advising apparatus measures body fat. Impedance in the body is calculated based on electric potential received from both electrodes for measuring voltage. A measurement starting switch is provided in a tip portion of the electrode for applying current on the right-thumb side. The measurement starting switch serves as a trigger for receiving electric potential from both electrodes for measuring voltage. When both hands are placed in contact with the corresponding electrodes for applying current and electrodes for measuring voltage, the right thumb naturally comes into contact with the measurement starting switch. Measurement of electric potential by the electrodes for measuring voltage is then started.

However, the health management index advising apparatus described in JP-A-H10-174680 merely measures the impedance in the body once. On the other hand, the blood pressure measuring device detects electrocardiographic signals and pulse wave signals that continuously change, and calculates blood pressure from the detected values. Noise tends to corrupt electrocardiograms and the pulse waves depending on changes in the state of contact with the electrodes and the pulse wave sensor. Measurement becomes difficult in many cases. In other words, compared to the above-described health management index advising apparatus, there is a stronger demand for the blood pressure measuring device to have an operating system that allows measurement to be started while contact is maintained with the electrodes and the pulse wave sensor. In the above-described conventional configuration, no consideration was given to the necessity of favorably maintaining a state of contact with not only the electrodes but also the pulse wave sensor.

### SUMMARY

The present disclosure provides a blood pressure measuring device capable of actualizing an operating system that allows acquisition of measurement information required for calculation of blood pressure to be started while a casing configuring the blood pressure measuring device is being held.

According to one aspect of the present disclosure, there is provided a blood pressure measuring device including: a casing that is held in both hands; a pair of electrocardiographic electrodes respectively provided such as to allow contact with the hands holding the casing and which detect electrocardiographic signals through the hands; a pulse wave sensor provided such as to allow contact with either of the hands holding the casing and which detects pulse wave signals through the hand; a measuring section that acquires measurement information including (i) a time difference between an electrocardiographic R wave based on the electrocardiographic signals and a pulse wave reference point based on the pulse wave signals, and (ii) a pulse wave amplitude based on the pulse wave signals; a calculating section that calculates blood pressure using the measurement information; a display section that displays the blood pressure; and a measurement starting section configured to enable the measuring section to start acquisition of the measurement information in a state in which contact is maintained between (a) the hands holding the casing and (b) the corresponding electrocardiographic electrodes and pulse wave sensor.

In the blood pressure measuring device of the present disclosure, acquisition of measurement information required for calculation of blood pressure is started while the user is holding the casing of the blood pressure measuring device in both hands and contact is maintained between the hands and the corresponding electrocardiographic electrodes and pulse wave sensor. In other words, the palms and fingers holding the casing do not need to be repositioned when the measuring section starts acquisition of the measurement information. Therefore, electrocardiographic signals and pulse wave signals can be measured with high accuracy. Accurate blood pressure can be obtained in a short amount of time.

### BRIEF DESCRIPTION OF DRAWINGS

In the accompanying drawings:

FIG. 1 is a perspective view of a blood pressure measuring device according to a first exemplary embodiment;

FIG. 2A is a front view of the blood pressure measuring device in FIG. 1;

FIG. 2B is a planar view of FIG. 2A;

FIG. 2C is a side view of FIG. 2A;

FIG. 2D is a rear view of FIG. 2A;

FIG. 2E is a bottom view of FIG. 2A;

FIG. 3A is a front view of a state in which the blood pressure measuring device in FIG. 1 is held in both hands;

FIG. 3B is a planar view of FIG. 3A;

FIG. 4 is an explanatory diagram of an operating system of the blood pressure measuring device in FIG. 1;

FIGS. 5A and 5B are explanatory diagrams of the blood pressure measuring device in FIG. 1, showing a state of use;

FIG. 6 is a state transition diagram of the blood pressure measuring device in FIG. 1;

FIG. 7 is a flowchart of a blood pressure calculation program run by a measurement circuit and a calculation circuit, corresponding with FIG. 6;

FIG. 8A is a graph indicating an electrocardiogram and pulse waves;

FIG. 8B is a graph indicating pulse waves, and FIG. 8C is a graph indicating acceleration pulse waves;

FIG. 9A is a front view of a blood pressure measuring device in a first variation example according to the first exemplary embodiment;

FIG. 9B is a rear view of a blood pressure measuring device in a second variation example according to the first exemplary embodiment;

FIG. 10A is a front view of a blood pressure measuring device in a third variation example according to the first exemplary embodiment;

FIG. 10B is a front view of a blood pressure measuring device in a fourth variation example according to the first exemplary embodiment;

FIG. 11 is a state transition diagram of a blood pressure measuring device in a fifth variation example according to the first exemplary embodiment;

FIG. 12 is a flowchart of a blood pressure calculation program run by a measurement circuit and a calculation circuit, corresponding with FIG. 11;

FIG. 13 is a front view of a blood pressure measuring device in a sixth variation example according to the first exemplary embodiment;

FIG. 14 is a state transition diagram of the blood pressure measuring device in FIG. 13;

FIG. 15 is a flowchart of a blood pressure calculation program run by a measurement circuit and a calculation circuit, corresponding with FIG. 14;

FIGS. 16A and 16B are explanatory diagrams of a blood pressure measuring device according to a second exemplary embodiment, showing a state of use;

FIG. 17 is an explanatory diagram of a blood pressure measuring device in a third variation example according to the second exemplary embodiment, showing a state of use;

FIG. 18 is an explanatory diagram of a blood pressure measuring device according to a third exemplary embodiment, showing a state of use;

FIG. 19 is an explanatory diagram of a blood pressure measuring device according to a fourth exemplary embodiment, showing a state of use;

FIG. 20 is an explanatory diagram of a blood pressure measuring device according to a fifth exemplary embodiment, showing a state of use; and

FIG. 21 is an explanatory diagram of a blood pressure measuring device in a first variation example according to the fifth exemplary embodiment, showing a state of use.

### DESCRIPTION OF EMBODIMENTS

Embodiments of the present invention will hereinafter be described with reference to the drawings.

### First Exemplary Embodiment

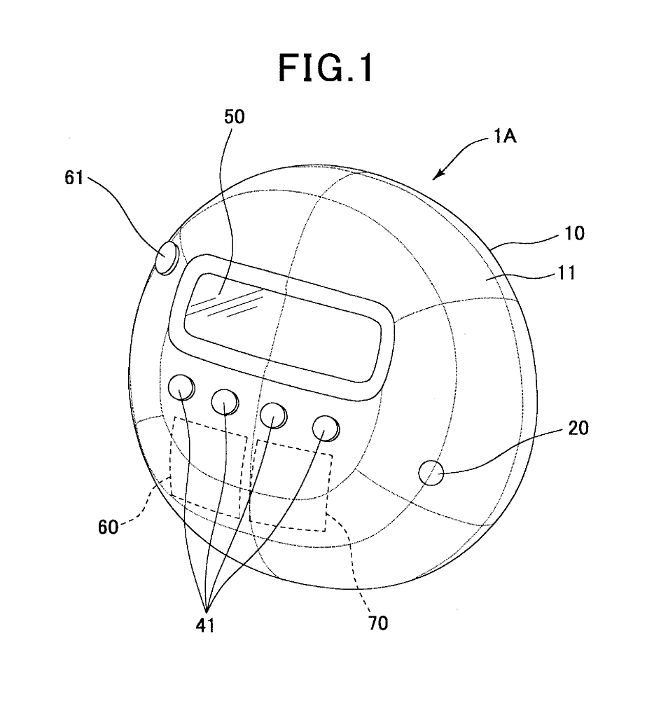

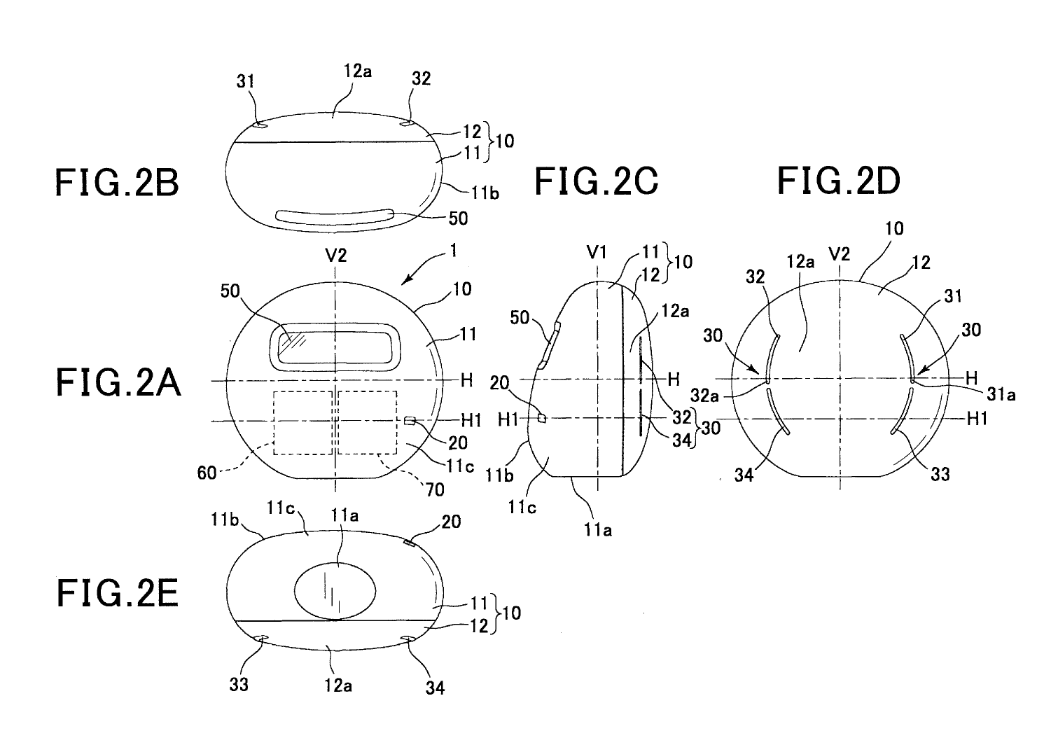

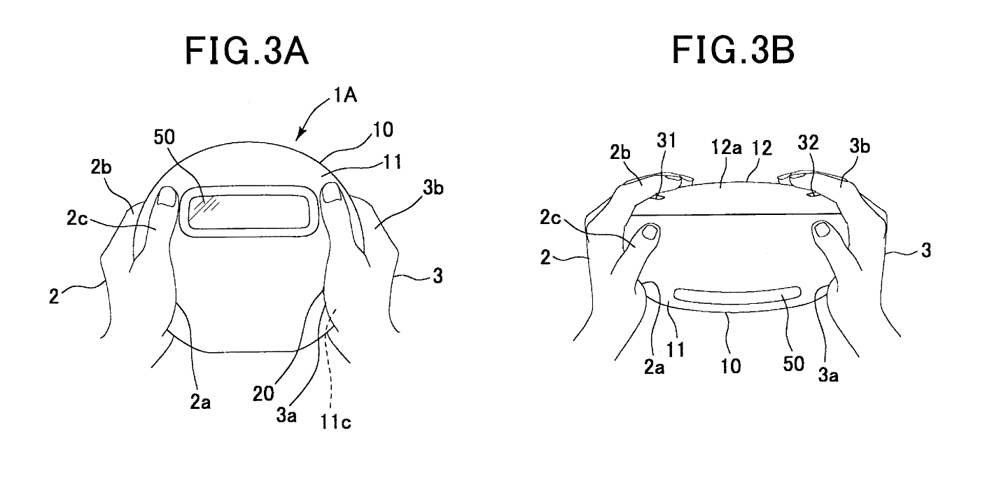

A blood pressure measuring device 1A provides a function for detecting electrocardiographic signals and pulse wave signals of a user (measurement subject), a function for acquiring measurement information based on the electrocardiographic signals and pulse wave signals, a function for calculating blood pressure using the measurement information, and a function for displaying the calculated blood pressure. As shown in FIG. 1 to FIG. 3A and FIG. 3B, the blood pressure measuring device 1A includes a casing 10, a pulse wave sensor 20, electrocardiographic electrodes 30, information input buttons 41, a display section 50, a measurement circuit 60, an operating button 61, and a calculation circuit 70. Illustration of the operating system (manipulating system), such as the information input buttons 41 and the operating button 61, is omitted in FIG. 2A to FIG. 2E, FIG. 3A and FIG. 3B.

The casing 10 is formed into a spherical shape. Here, “spherical shape” in the present specification is not limited to a sphere in the strict sense of the geometric definition. Rather, “spherical shape” widely includes shapes of which the cross-sectional shape (horizontal cross-section or vertical cross-section) is oval or elliptical, a smooth, convex, closed curve similar to an ellipse, an oblong shape, and the like. Specifically, the casing 10 is substantially circular from a front view (see FIG. 2A), substantially elliptical from a planar view (see FIG. 2B), and substantially oval from a side view (see FIG. 2C). The casing 10 includes a case body 11 and a cover body 12. The casing 10 stands when placed on a surface with a flat bottom portion 11a of the case body 11 on the bottom side.

The case body 11 and the cover body 12 together house a circuit board therein (see broken lines in FIG. 1 and FIG. 2A). The circuit board includes the measurement circuit 60 and the calculation circuit 70. The case body 11 is located on the front side and the cover body 12 is located on the rear side (see FIG. 2B and FIG. 2C). Specifically, when a horizontal plane bisecting the casing 10 in the height direction is H and a vertical plane bisecting the casing 10 in the front/rear thickness direction set by the horizontal plane H is V1, the cover body 12 is placed further towards the rear than the vertical plane V1. In addition, when a vertical plane bisecting the casing 10 in the left/right thickness direction set by the horizontal plane H is V2, the casing 10 is symmetrical in relation to the vertical plane V2.

As shown in the side view in FIG. 2C, the case body 11 has a peak portion 11b that projects furthest towards the front side in an area below the horizontal plane H. A peripheral surface 11c below the peak portion 11b is formed into a curved shape that is similar to the natural curve of the user's palm in a relaxed state. The normal line of the curved shape extends downward at an angle. Therefore, as shown in FIG. 3A and FIG. 3B, when the user holds the casing 10 in both hands 2 and 3, it is natural for the case body 11 to be supported and held from below at an angle (laterally at an angle) with the palms 2a and 3a in close contact with the peripheral surface 11c of the case body 11.

In other words, when the user holds the casing 10 in both hands 2 and 3, the palms 2a and 2b and the base portions of the fingers 2b and 3b come into surface contact with the peripheral surface 11c of the case body 11, and the tip portions and middle portions of the fingers 2b and 3b come into surface contact with a peripheral surface 12a of the cover body 12, based on the natural bending movement of the palms 2a and 3a and the fingers 2b and 3b. Therefore, the user does not grasp the casing 10 in a grip. The hands 2 and 3 are not excessively flexed, and stable measurement can be performed.

The pulse sensor 20 is a known reflective optical sensor including a light-emitting element (such as a light-emitting diode) and a light-receiving element (such as a photodiode). Specifically, when light is emitted from the light-emitting element towards the user's hand, some of the light is absorbed by the hemoglobin in the blood flowing through the arterioles (capillaries) running through the human body. The remaining light is reflected by the arterioles and dispersed. Some of the dispersed light enters the light-receiving element. The amount of hemoglobin flowing through the arterioles changes in a pulsing manner as a result of pulsation of the blood. The light absorbed by the hemoglobin also changes in a pulsing manner. Therefore, the amount of received light reflected by the arterioles and detected by the light-receiving element changes. The changes in the amount of received light at this time are outputted to the measurement circuit 60 as the pulse wave signals (such as voltage signals).

The pulse wave sensor 20 is provided in correspondence with one hand 3 holding the casing 10. Specifically, the pulse wave sensor 20 is located below the horizontal plane H and before the vertical plane V1. More specifically, as shown in the side view in FIG. 2C, the pulse wave sensor 20 is placed in a position above the peak portion 11b of the case body 11 that projects furthest towards the front side, near the peak portion 11b. The pulse wave sensor 20 is embedded in the section corresponding to the thenar eminence of the palm 3a (the ball or fleshy portion at the base of the thumb) when the case 10 is held in both hands 2 and 3 (see FIG. 3A and FIG. 3B). The position of the pulse wave sensor 20 is not limited to the section corresponding to the thenar eminence of the palm 3a. The pulse wave sensor 20 may be provided in a section corresponding to the thenar area of the palm 2a.

When the pulse wave signals are detected by the pulse wave sensor 20, the pulse wave sensor 20 is required to be pressed with suitable force. In other words, an amount of force ensuring that the pulse wave sensor 20 and the skin of the palm 3a (2a) are in contact with certainty is required to be applied to the pulse wave sensor 20. However, if too much force is applied, the blood vessels under the skin become crushed and the pulse wave signals cannot be detected. On the other hand, if the portion of the hand pressing the pulse wave sensor 20 is the thumb, although force can be adjusted so as not to be too strong, the thumb is required to be placed on the pulse wave sensor 20. In addition, because a constant and suitable amount of force is required to be continuously applied to the pulse wave sensor 20, the thumb tends to shake, thereby easily causing noise.

On the other hand, when the pulse wave sensor 20 is provided in the section corresponding to the thenar eminence of the palm 3a (2a), the pulse wave sensor 20 and the palm 3a (2a) can be placed in contact with each other simply by the casing 10 being held in the hands 2 and 3. The user is not required to be aware of the presence of the pulse wave sensor 20. Therefore, a constant and suitable amount of force can be continuously applied to the pulse wave sensor 20.

The electrocardiographic electrodes 20 detect the electrocardiographic signals (signals based on the potential difference between electrodes) and output the detected electrocardiographic signals to the measurement circuit 60. As shown in FIG. 2C and FIG. 2D, the electrocardiographic electrodes 20 include a left-hand electrode 31 and a right-hand electrode 32 forming a pair to the left and right provided in correspondence with the fingers 2b and 3b of the hands 2 and 3 holding the casing 10, and intermediate electrodes 33 and 34 that similarly form a pair. The electrodes 31 to 34 are assembled within the casing 10 such as to project from the cover body 12 by a predetermined amount (such as 0.5 mm to 1.0 mm).

When a plane that passes through the pulse wave sensor 20 and is parallel with the horizontal plane H is a horizontal plane H1, the left-hand electrode 31 and the right-hand electrode 32 are located such that at least a portion of each is above the horizontal plane H1. This is so that, when the user holds the casing 10 in both hands 2 and 3 and supports the casing 10 from below at an angle (laterally at an angle) with the palms 2a and 3a in close contact with the peripheral surface 11c of the case body 11, the fingers 2b come into contact with the left-hand electrode 31 with certainty and the fingers 3b come in to contact with the right-hand electrode 32 with certainty, even when the holding positions of the palms 2a and 3a significantly change.

In a manner similar to the electrodes 31 and 32, the intermediate electrodes 33 and 34 are located such that at least a portion of each is above the horizontal plane H1. As a result, when the user holds the casing 10 in both hands 2 and 3 and supports the casing 10 from below at an angle (laterally at an angle) with the palms 2a and 3a in close contact with the peripheral surface 11c of the case body 11, the fingers 2b come into contact with the left-hand electrode 31 and the intermediate electrode 33 with certainty, and the fingers 3b come in to contact with the right-hand electrode 32 and the intermediate electrode 34 with certainty. The intermediate electrodes 33 and 34 are short-circuited inside the casing 10 and function as a single electrode. As a result of the signals detected by the electrodes 31 to 34 being, for example, operationally amplified by an operational amplifier, noise caused by body movement can be effectively removed.

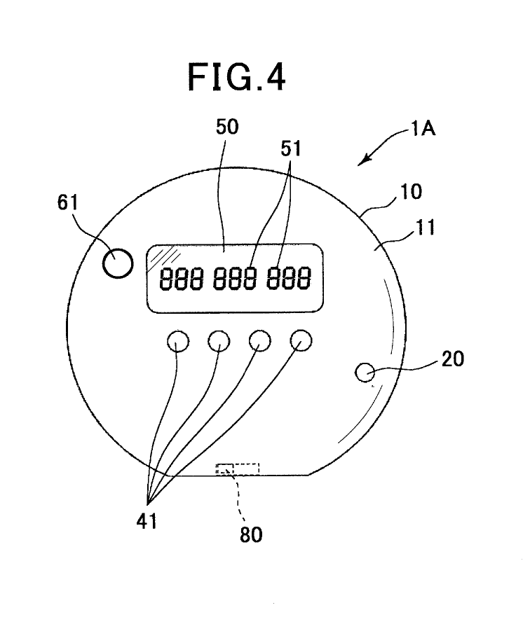

As shown in FIG. 4, the display section 50 is, for example, a display using a seven-segment light-emitting diode (LED) 51. The display section 50 is located above the horizontal plane H and before the vertical plane V1 to facilitate visibility by the user. The display section 50 displays measurement results, or in other words, the blood pressure (systolic blood pressure and diastolic blood pressure) calculated by the calculation circuit 70. The display section 50 is not limited to that using the seven-segment LED 51. Displays using liquid crystal display (LCD), organic electroluminescence (EL), and the like can be widely used.

The measurement circuit 60 includes a microcomputer, an input interface (I/F) circuit, and an output I/F circuit as main constituent components. The microcomputer is composed of a central processing unit (CPU), a read-only memory (ROM), a random access memory (RAM), and the like. The measurement circuit 60 acquires (calculates) measurement information required for calculation of blood pressure, based on the electrocardiographic signals from the electrocardiographic electrodes 30 and the pulse wave signals from the pulse wave sensor 20. The measurement information includes a time difference between an electrocardiographic R wave and a pulse wave reference point, shape parameters of the pulse waves including pulse wave amplitude, shape parameters of acceleration pulse waves including the amplitude of each peak in acceleration pulse waves that are second-order-differentiated pulse waves, and the like. The measurement circuit 60 stores the calculated values in the RAM and outputs the calculated values to the calculation circuit 70. The measurement circuit 60 is equivalent to a measuring section of the present invention.

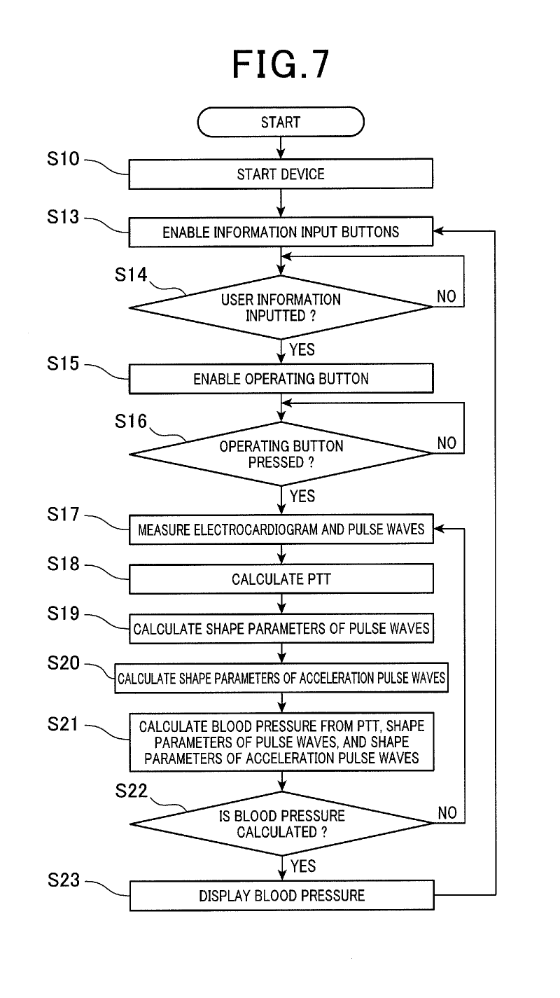

The calculation circuit 70 includes a microcomputer, an input I/F circuit, and an output I/F circuit as main constituent components. The microcomputer is composed of a CPU, a ROM, a RAM, and the like. The calculation circuit 70 calculates the blood pressure using the measurement information from the measurement circuit 60, in adherence to execution of a blood pressure calculation program shown in FIG. 7 that is stored in the ROM. The calculation circuit 70 stores the calculated values in the RAM and displays the calculated values in the display section 50. The calculation circuit 70 is equivalent to a calculating section of the present invention.

Next, an operating system of the blood pressure measuring device 1A will be described. According to the first exemplary embodiment, as shown in FIG. 4, the case body 11 is provided with a power switch 80, the information input buttons 41, and the operating button 61 as the operating system. The power switch 80 is a manually operated switch used to turn the measurement circuit 60 and the calculation circuit 70 (see FIG. 1 and FIG. 2A) ON and OFF. The information input buttons 41 are manually operated switches used to input user information, such as height, age, weight, and sex. The information input buttons 41 function as an information input section of the present invention.

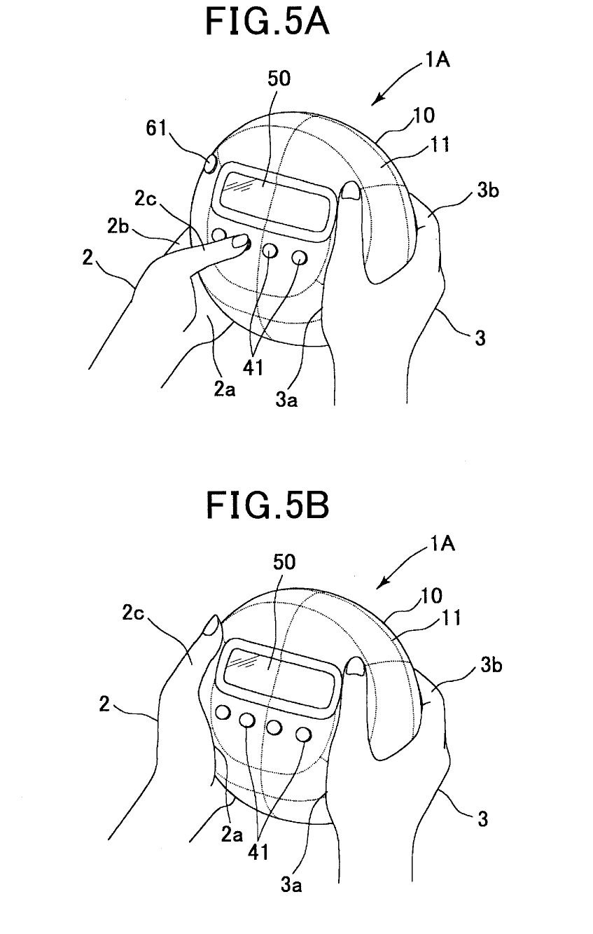

The operating button 61 is a manually operated switch serving as a trigger by which the measurement circuit 60 acquires the above-described measurement information. The operating button 61 is located in a position allowing contact with the finger 2b of the hand 2 that is not in contact with the pulse wave sensor 20, of the hands 2 and 3 holding the casing 10. Specifically, the operating button 61 is located in a position allowing operation by the thumb 2c of the hand 2 (see FIG. 5B), or in other words, in a section of the casing 10 above the horizontal plane H and before the vertical plane V1 (front surface section in the upper half of the casing 10).

Because the operating button 61 is provided in this way in a position visible to the user from the front view of the casing 10, the user can easily check whether or not the operating button 61 has been pressed. The user can feel a sense of ease regarding operation, and operability can be favorably ensured. The operating button 61 is equivalent to a measurement starting section of the present invention.

The blood pressure measuring device 1A according to the first exemplary embodiment can be used in the manner shown in FIG. 5A and FIG. 5B. First, the user touches the pulse wave sensor 20 with the palm 3a while supporting the casing 10 with the right hand 3. In a state in which the fingers 3b are in contact with the right-hand electrocardiographic electrode 32 and the intermediate electrode 34, the user inputs user information by operating the information input buttons 41 with the left hand 2 (FIG. 5A).

Then, the user places the left hand 2 on the casing 10 such as to support the casing 10 with both hands (FIG. 5B). At this time, the fingers 2b of the left hand 2 come into contact with the left-hand electrocardiographic electrode 31 and the intermediate electrode 33. During the flow of this series of movements of the left hand 2, the user presses the operating button 61 with the thumb 2c of the left hand 2. As a result, because the right hand 3a does not need to be repositioned at all after the palm 3a has come into contact with the pulse wave sensor 20, noise does not easily corrupt the pulse wave signals detected by the pulse wave sensor 20.

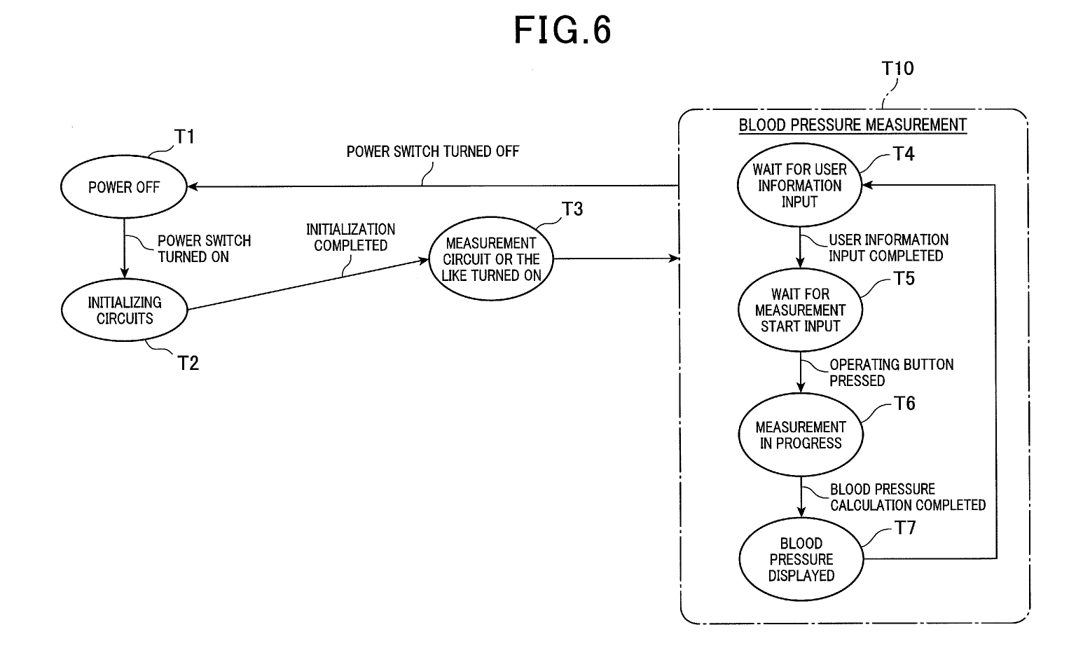

Next, operations of the blood pressure measuring device 1A configured as described above will be described. First, state transition of the blood pressure measuring device 1A will be described with reference to FIG. 6. As a result of the power switch 80 being turned ON from OFF (state T1), after initialization of the circuits (state T2), the measurement circuit 60 and the calculation circuit 70 (see FIG. 1 and FIG. 2A) are turned ON (state T3). The blood pressure measuring device 1A transitions to blood pressure measurement mode (state T10) and waits for user information input (state T4). In this state, when user information input is completed, the blood pressure measuring device 1A waits for measurement start input (state T5). When the operating button 61 is operated by being pressed, the blood pressure measuring device 1A transitions to “measurement-in-progress” mode and starts acquisition of measurement information (state T6). When calculation of blood pressure based on the measurement information is completed, the blood pressure is displayed in the display section 50 (state T7). The blood pressure measuring device 1A subsequently waits for user information input (state T4). When user information input is performed, the blood pressure measuring device 1A enters the states T5 to T7. On the other hand, the blood pressure measuring device 1A stops running if the power switch 80 is turned OFF (state T1).

Next, the operations of the blood pressure measuring device 1A will be described in further detail, based on the blood pressure calculation program shown in FIG. 7. The blood pressure measuring device 1A is started when the power switch 80 is turned ON (step S10). Input operation by the information input buttons 41 is enabled (step S13). When user information is inputted by operation of the information input buttons 41 (YES at step S14), input operation by the operating button 61 is enabled (step S15). In other words, when user information has not been inputted (NO at step S14), input operation by the operating button 61 is disabled. With the press-operation of the operating button 61 as a trigger, the measurement circuit 60 performs processing operations at steps S17 to S20 and the calculation circuit 70 performs processing operations at steps S21 to S23.

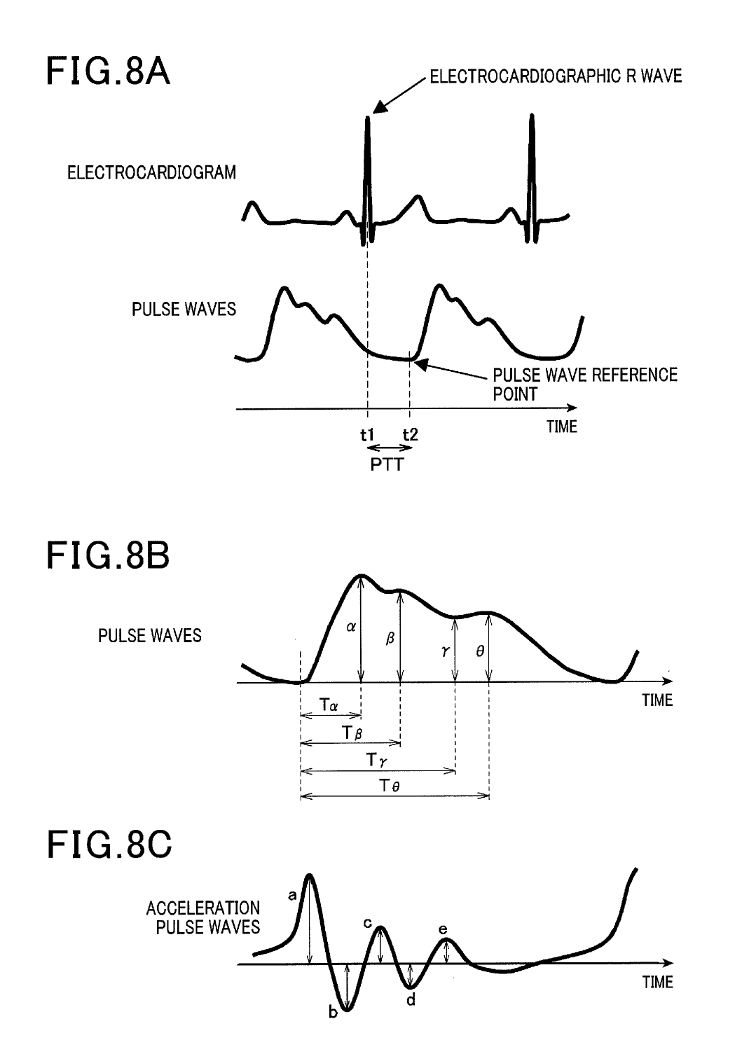

Specifically, as shown in FIG. 8A, the measurement circuit 60 measures an electrocardiogram based on the signals detected by the electrocardiographic electrodes 30, and measures the pulse waves based on the signals detected by the pulse wave sensor 20 (step S17). Then, the measurement circuit 60 calculates a time difference PTT (a time delay (t2-t1) of the pulse wave signal in relation to the electrocardiographic signal) between an electrocardiographic R wave in the electrocardiogram and a pulse wave reference point in the pulse waves (such as a rising point) (step S18). The measurement circuit 60 also calculates shape parameters (α, β, γ, θ, T_{α}, T_{β}, T_{γ}, T_{θ}, . . . ) of the pulse waves such as those shown in FIG. 8B (step S19), and calculates shape parameters (a, b, c, d, e) of the acceleration pulse waves that are second-order-differentiated pulse waves such as those shown in FIG. 8C (step S20).

Next, the calculation circuit 70 determines blood pressure BP using the time difference PTT, the shape parameters (α, β, γ, θ, T_{α}, T_{β}, T_{γ}, T_{θ}, . . . ) of the pulse waves, and the shape parameters (a, b, c, d, e) of the acceleration pulse waves from the measurement circuit 60, and further taking into consideration the user information, by a following formula (I).

BP=F(PTT, α, β, γ, θ, T_{α}, T_{β}, T_{γ}, T_{θ}, a, b, c, d, e, . . . ) (1)

After performing the processing operation at Step S21, the calculation circuit 70 displays the determined blood pressure BP in the display section 50 via the output I/F circuit (YES at step S22, step S23). In this instance, in a manner similar to typical blood pressure gauges, the systolic blood pressure and the diastolic blood pressure are displayed as the blood pressure BP.

As is clear from the description above, according to the first exemplary embodiment, the user holds the casing 10 of the blood pressure measuring device 1A in both hands 2 and 3. In a state in which contact between each hand and its corresponding electrocardiographic electrodes 30 (31 to 34) and the pulse wave sensor 20 is maintained, acquisition of measurement information required for calculation of blood pressure is started. In other words, when the measurement circuit 60 starts acquisition of the measurement information, the palm 3a and the fingers 3b holding the casing 10 are not required to be repositioned. Therefore, the electrocardiographic signals and the pulse wave signals can be measured with high accuracy. Accurate blood pressure can be obtained in a short amount of time.

### Variation Examples

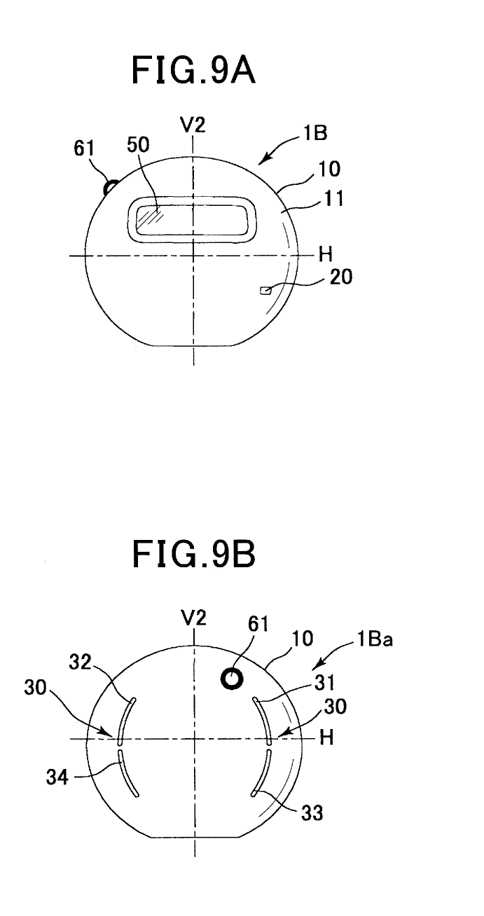

According to the above-described first exemplary embodiment, the operating button 61 is located in a position allowing operation by the thumb 2c of the hand 2 not in contact with the pulse wave sensor 20 (see FIG. 5B), of the hands 2 and 3 holding the casing 10. In other words, the operating button 61 is located above the horizontal plane H and before the vertical plane V1 in the casing 10. However, instead, the operating button 61 may be located in a position allowing operation by the pointer finger 2b of the hand 2 not in contact with the pulse wave sensor 20, of the hands 2 and 3 holding the casing 10, such as above the horizontal plane H and on the side opposite to the pulse wave sensor 20 with reference to the vertical plane V2 in the casing 10 (first variation example) as shown, for example, in a blood pressure measuring device 1B in FIG. 9A. Alternatively, the operating button 61 may be located above the horizontal plane H and behind the vertical plane V1 (see FIG. 2C) as shown, for example, in a blood pressure measuring device 1Ba in FIG. 9B (second variation example).

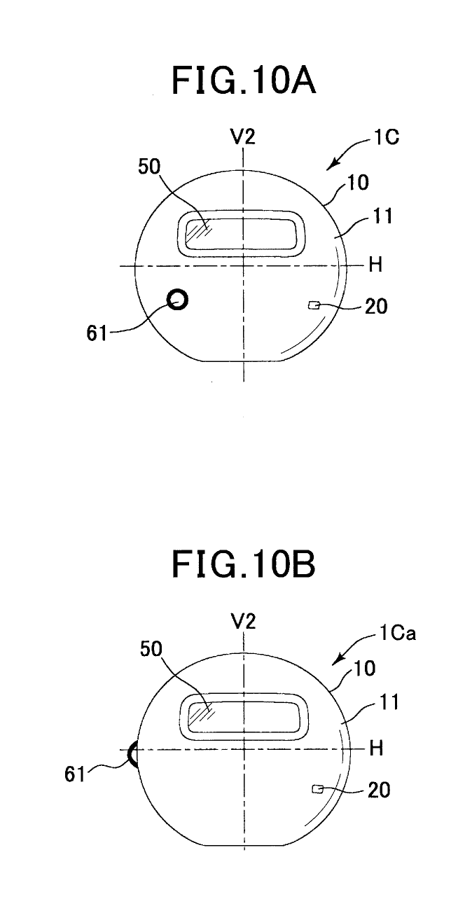

In addition, the operating button 61 is not limited to that located in a position allowing operation by the pointer finger of the hand 2 not in contact with the pulse wave sensor 20 as in the above-described first variation example. The operating button 61 may be located in a position allowing operation by the palm 2b not in contact with the pulse wave sensor 20, of the hands 2 and 3 holding the casing 10, such as below the horizontal plane H and before the vertical plane V1 in the casing 10 as shown, for example, in a blood pressure measuring device 1C in FIG. 10A (third variation example). Alternatively, the operating button 61 may be located on the side opposite to the pulse wave sensor 20 with reference to the vertical plane V2 in the casing 10 as shown, for example, in a blood pressure measuring device 1Ca in FIG. 10B (fourth variation example). In FIG. 9A, FIG. 9B, FIG. 10A, and FIG. 10B, illustration of the information input buttons 41 is omitted. Other configurations are the same as those according to the above-described first exemplary embodiment. Therefore, components and sections corresponding with those according to the first exemplary embodiment are given the same reference numbers. Explanations thereof are omitted.

As a result of the first to fourth variation examples as well, in a manner similar to that according to the first exemplary embodiment, when the measurement circuit 60 starts acquisition of the measurement information, the palm 3a and the fingers 3b holding the casing 10 are not required to be repositioned. Therefore, the electrocardiographic signals and the pulse wave signals can be measured with high accuracy.

In addition, according to the first exemplary embodiment and in the first to fourth variation examples, the information input buttons 41 for inputting user information are provided in the casing 10. The blood pressure is calculated taking into consideration the user information inputted by the information input buttons 41. Therefore, the blood pressure can be accurately calculated. However, the blood pressure can also be simply calculated without use of the user information. In this instance, a configuration omitting the information input buttons 41 (fifth variation example [applicable to the first exemplary embodiment and each of the first to fourth variation examples]) is also possible.

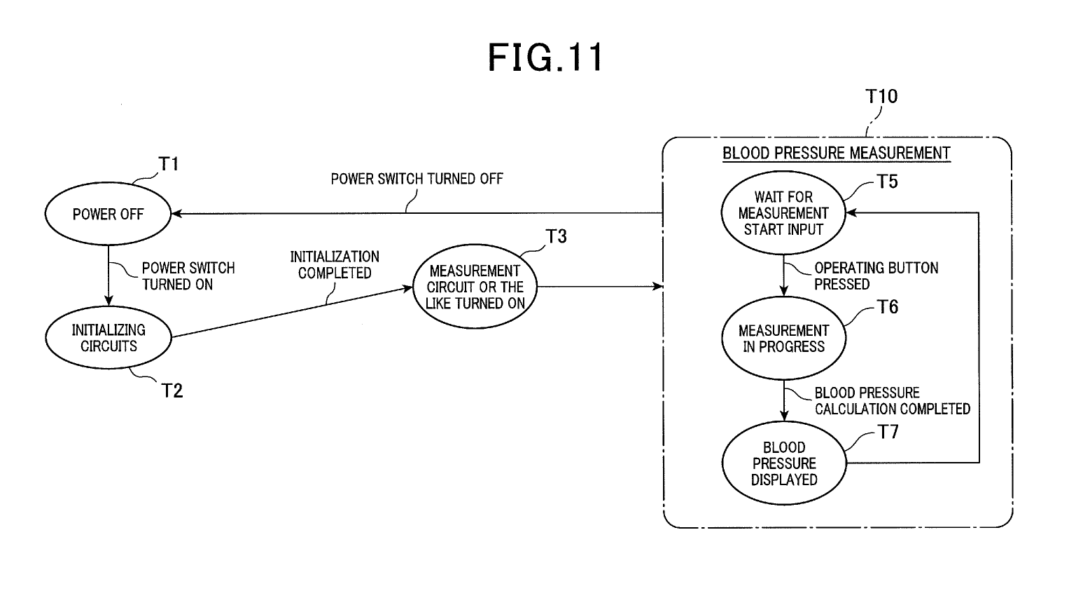

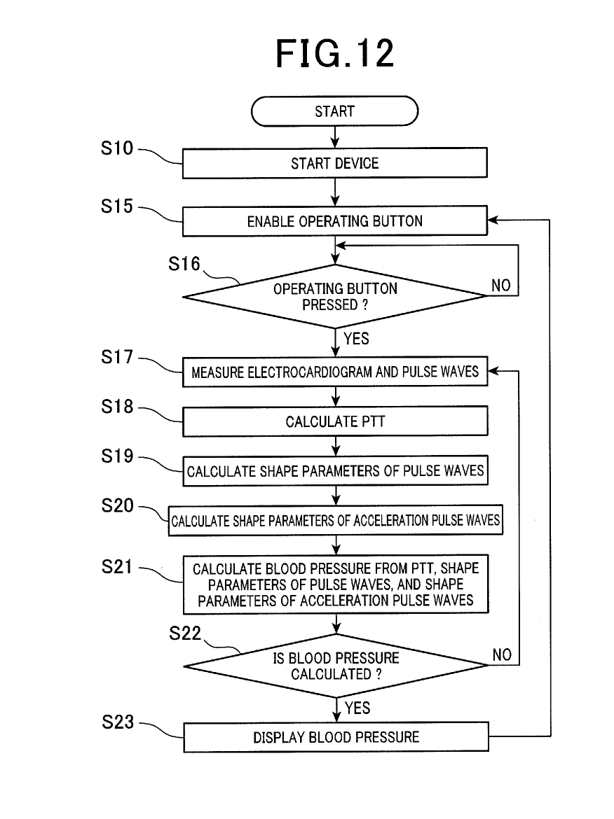

In the fifth variation example, as shown in FIG. 11, after the measurement circuit 60 and the calculation circuit 70 are turned ON (state T3), in the blood pressure measurement mode (state T10), the blood pressure measuring device immediately waits for measurement start input (state T5) without waiting for user information input (state T4 in FIG. 6). In correspondence with the wait for user information input being omitted, in the blood pressure calculation program shown in FIG. 12 that is executed in the fifth variation example, the processing operations at Step S13 and Step S14 are omitted from the blood pressure calculation program shown in FIG. 7 that is executed according to the first exemplary embodiment. The other steps in the blood pressure calculation program are the same as those according to the first exemplary embodiment. Therefore, corresponding steps are given the same reference numbers. Explanations thereof are omitted.

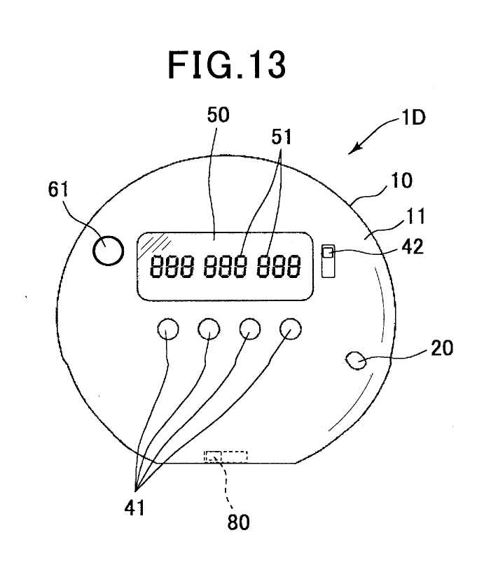

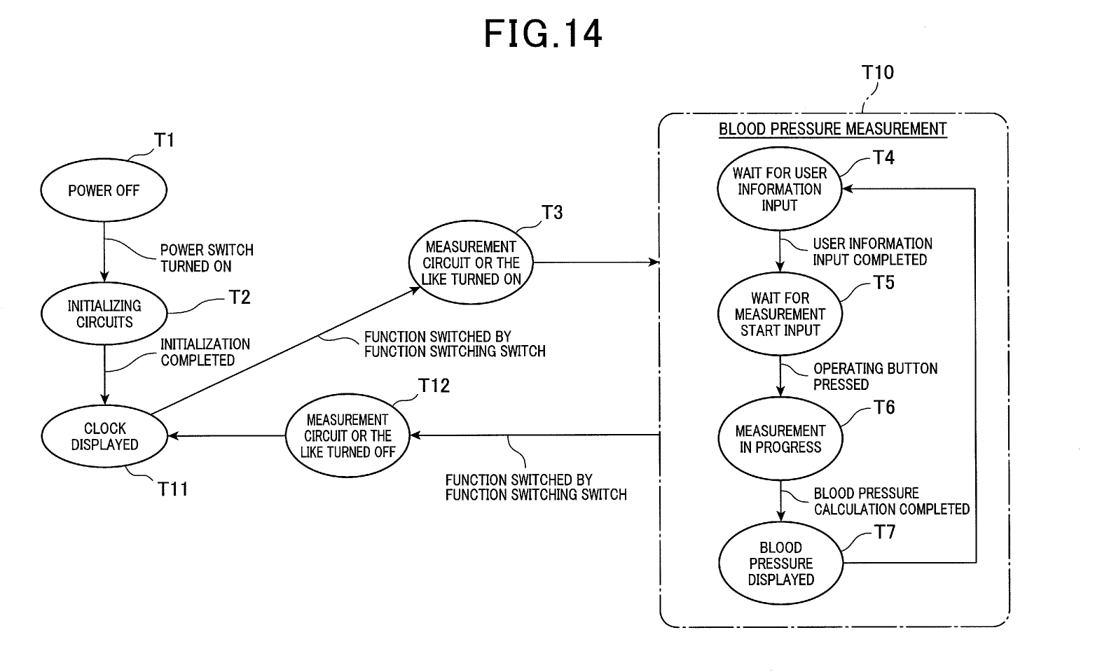

The blood pressure measuring device 1A and the like according to the first exemplary embodiment and in the first to fifth variation examples may be provided with a clock display function. FIG. 13 shows the blood pressure measuring device 1A according to the first exemplary embodiment provided with a clock display function (sixth variation example) as a representative example. As shown in FIG. 14, in a blood pressure measuring device 1D in the sixth variation example, as a result of the power switch 80 being turned ON from OFF (state T1), after initialization of the circuits (state T2), the clock display function is activated and the time is displayed in the display section (state T11). When the user wants to measure blood pressure, the user operates a function switching switch 42 provided in the casing 10, and switches the blood pressure measuring device 1D from clock display mode to blood pressure measurement mode (state T10). When the blood pressure display device 1D is switched to clock display mode, the blood pressure display device 1D is in a power saving state in which the measurement circuit 60 and the calculation circuit 70 are turned OFF (state T12). When the blood pressure display device 1D is switched to blood pressure measurement mode (state T10), the measurement circuit 60 and the calculation circuit 70 are turned ON (state T3). After measurement of blood pressure is completed, the user operates the function switching switch 42 and the blood pressure measuring device 1D is switched from blood pressure measurement mode (state T10) to clock display mode.

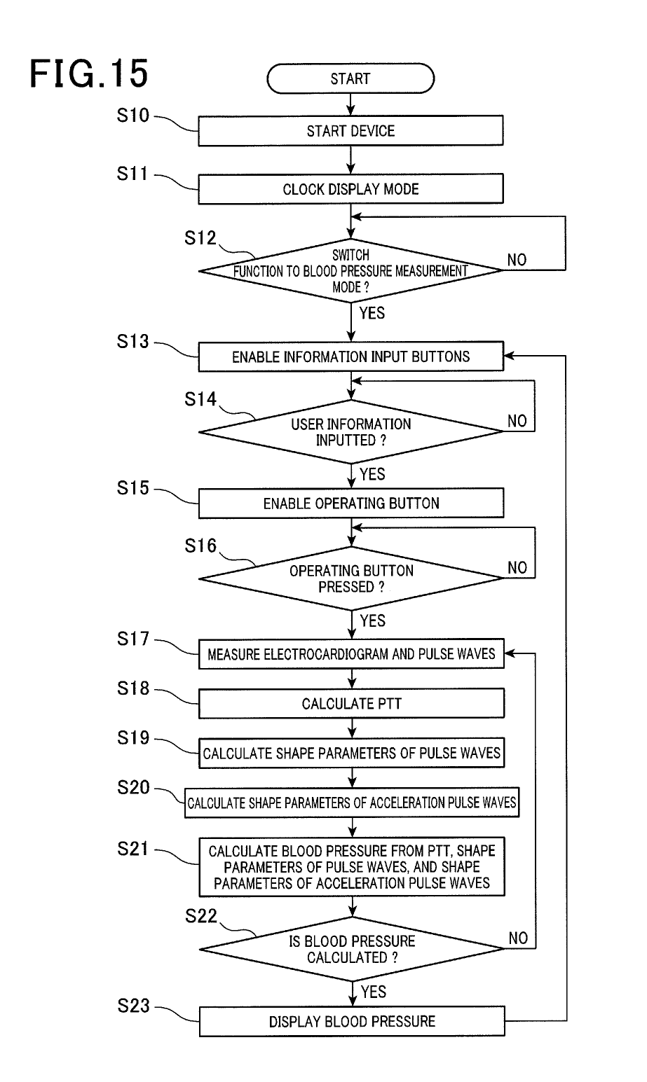

In correspondence with the addition of the clock display function, in the blood pressure calculation program shown in FIG. 15 that is executed in the sixth variation example, processing operations at steps S11 and S12 are added to the blood pressure calculation program shown in FIG. 7 that is executed according to the first exemplary embodiment. When the blood pressure measuring device 1D is switched to blood pressure measurement mode by the function switching switch 42 being operated (YES at step S12), the processing operations at step S13 and subsequent steps (processing operations at step S15 and subsequent steps if the sixth variation example is applied to the fifth variation example in which the information input buttons 14 are omitted) are performed. The blood pressure measuring device 1D is set to immediately transitions to clock display mode when the user operates the function switching switch 42 during blood pressure measurement mode (steps S13 to S23) and switches to clock display mode. Other steps are the same as those according to the first exemplary embodiment. Corresponding steps are given the same reference numbers. Explanations thereof are omitted.

Application of the specification of the sixth variation example is not limited to the blood pressure measuring device 1A in which user information is inputted by the information input buttons 41. The sixth variation example can be applied to the blood pressure measuring device in the fifth variation example in which the information input buttons 41 are omitted from the blood pressure measuring device 1A. Furthermore, the sixth variation example can be applied to the blood pressure measuring devices 1B, 1Ba, 1C, and 1Ca in the first to fourth variation examples in which the position of the operating button 61 differs, and respective blood pressure measuring devices corresponding with the fifth variation example in which the information input buttons 41 are omitted from the blood pressure measuring devices 1B, 1Ba, 1C, and 1Ca.

According to the first exemplary embodiment and in the first to sixth variation examples, the pulse wave sensor 20 is provided in a position allowing contact with the palm 3a or the finger 3b of the right hand 3. The operating button 61 is provided in a position allowing contact with the palm 2a, the pointer finger 2b, or the thumb 2c of the left hand 2. However, a configuration in which the pulse wave sensor 20 is in contact with the left hand 2 and the operating button 61 is in contact with the right hand 3 is also possible.

### Second Exemplary Embodiment

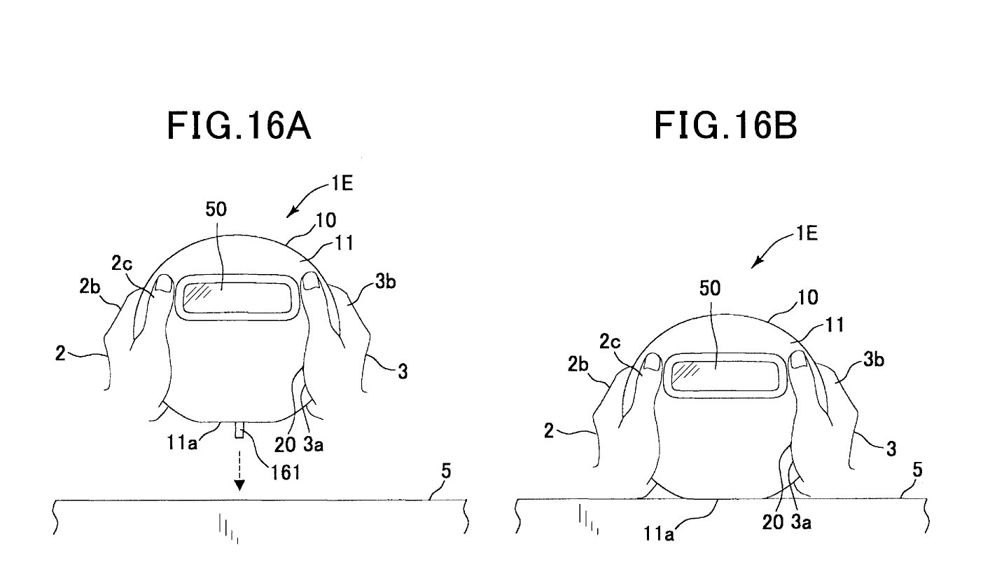

According to the above-described first exemplary embodiment, the operating button 61 is provided in a position allowing contact with the palm 2a, the pointer finger 2b, or the thumb 2c of the left hand 2. However, instead, a configuration is possible in which an operating button 161 is provided on the bottom surface 11a of the casing 10 as shown, for example, in a blood pressure measuring device 1E in FIG. 16. In FIG. 16, illustration of the information input buttons 41 is omitted. Other configurations are the same as those according to the first exemplary embodiment. Therefore, components and sections corresponding with those according to the first exemplary embodiment are given the same reference numbers. Explanations thereof are omitted.

The operating button 161 is biased in a direction projecting from the bottom surface 11a of the casing 10. When the casing 10 is placed on a surface 5 of a desk, a floor, or the like with the bottom surface 11a on the bottom side while the casing 10 is being held in both hands 2 and 3, the operating button 161 is pressed upwards by the surface 5 and retracts into the casing 10. In other words, the blood pressure measuring device 1E is turned OFF when the operating button 161 is projecting outside of the casing 10. On the other hand, when the operating button 161 retracts into the casing 10, the measurement circuit 60 and the calculation circuit 70 are turned ON. Acquisition of measurement information by the measurement circuit 60 is then started.

As a result of the casing 10 being placed on the surface 5, body movement by the user is suppressed. Noise accompanying body movement does not easily corrupt the electrocardiographic signals and the pulse wave signals. Therefore, according to the second exemplary embodiment, an effect is achieved in which blood pressure can be accurately calculated in a shorter amount of time.

### Variation Examples

According to the second exemplary embodiment as well, in a manner similar to that in the above-described fifth variation example according to the first exemplary embodiment, blood pressure can be calculated without use of the user information. A configuration in which the information input buttons 41 are omitted is possible (first variation example). In addition, in a manner similar to that in the above-described sixth variation example according to the first exemplary embodiment, the function switching switch 42 can be provided in the casing 10 (second variation example).

In the second variation example, the blood pressure measuring device can be set such that, when the function switching switch 42 is switched to clock display mode, the measurement circuit 60 and the calculation circuit 70 are turned OFF regardless of whether the operating button 161 is turned ON or OFF. On the other hand, the measurement circuit 60 and the calculation circuit 70 are turned ON under a condition that the function switching switch 42 is switched to blood pressure measurement mode and the operating button 161 is turned ON.

In other words, the blood pressure measuring device can be set such that acquisition of measurement information by the measurement circuit 60 is started when the intent of the user to perform blood pressure measurement is confirmed and a state in which noise accompanying body movement does not easily corrupt the electrocardiographic signals and the pulse wave signals is established. In the second variation example as well, in a manner similar to that according to the second exemplary embodiment, blood pressure can be accurately calculated in a shorter amount of time.

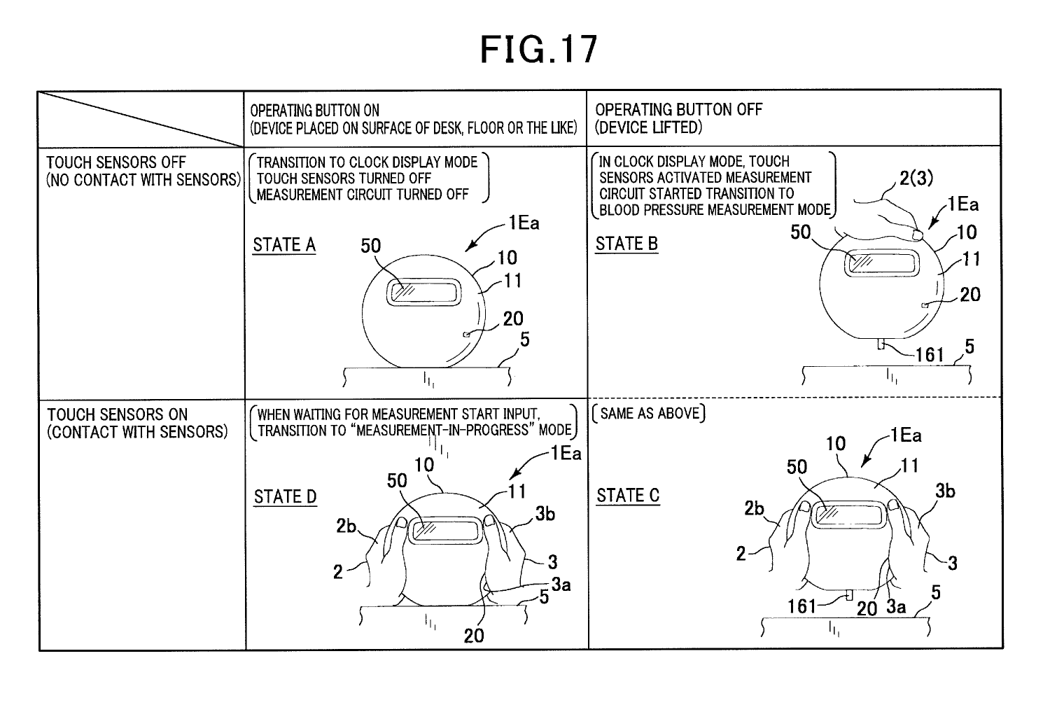

In the second variation example according to the second exemplary embodiment, at least a manual switching operation by the function switching switch 42 is required to transition the blood pressure measuring device 1E having the clock display function from clock display mode to blood pressure measurement mode. However, instead, a configuration is possible in which the transition between clock display mode and blood pressure measurement mode is automatically performed as shown, for example, in a blood pressure measuring device 1Ea in FIG. 17 (third variation example). In FIG. 17, illustration of the information input buttons 41 is omitted. Other configurations are the same as those according to the above-described second exemplary embodiment. Therefore, components and sections corresponding with those according to the second exemplary embodiment are given the same reference numbers. Explanations thereof are omitted.

In the third variation example, the pulse wave sensor 20 and the electrocardiographic electrodes 30 each also function as a touch sensor that detects contact made to the pulse wave sensor 20 or the electrocardiographic electrodes 30 itself. Contact made to the electrocardiographic electrodes 30 can be detected by, for example, the impedance between the electrocardiographic electrodes 31 and 32 being measured. On the other hand, contact made to the pulse wave sensor 20 can be detected by, for example, judgment being made that light to the light receiving section has been blocked by a palm when signals from the light receiving section fall to a predetermined threshold value or below. Alternatively, a movable section can be provided in the pulse wave sensor 20. The light to the light receiving section can be judged to be blocked by a palm when the movable section is pressed by the palm. In clock display mode, when the operating button 161 is turned from ON to OFF, the blood pressure measuring device 1Ea switches from clock display mode to blood pressure measurement mode. When the touch sensors are turned ON and the operating button 161 is turned from OFF to ON, acquisition of the measurement information is started.

Specifically, as shown in a state (A) of FIG. 17, when the blood pressure measuring device 1Ea is placed on the surface 5 and the operating button 161 is turned ON, the blood pressure measuring device 1Ea transitions to clock display mode. In clock display mode, power required for the pulse wave sensor 20 and the electrocardiographic electrodes 30 to function as touch sensors is turned OFF. The measurement circuit 60 is also turned OFF.

From this state, as shown in a state (B) of FIG. 17, when the blood pressure measuring device 1Ea is lifted and the operating button 161 is turned ON, the touch sensors are activated. In other words, the power required for the pulse wave sensor 20 and the electrocardiographic electrodes 30 to function as touch sensors is turned ON. The measurement circuit 60 is also turned ON. The blood pressure measuring device 1Ea transitions to blood pressure measurement mode.

Then, as shown in a state (C) of FIG. 17, as a result of the casing 10 being supported by both hands 2 and 3, the palm 3a of the right hand 3 touching the pulse wave sensor 20, the fingers 3b touching the right-hand electrocardiographic electrode 32 and the intermediate electrode 34, and the fingers 2b of the left hand 2 touching the left-hand electrocardiographic electrode 31 and the intermediate electrode 33, the touch sensors are turned ON. In this instance, when the blood pressure measuring device is a type in which user information is inputted, such as that according to the above-described first exemplary embodiment (see FIG. 6), when the user information is inputted by operation of the information input buttons 41, the blood pressure measuring device waits for measurement start input. On the other hand, when the blood pressure measuring device is a type in which user information is not inputted, such as that in the above-described fifth variation example according to the first exemplary embodiment (see FIG. 11), the blood pressure measuring device waits for measurement start input as a result of the touch sensors being turned ON.

Finally, as shown in a state (D) of FIG. 17, when the casing 10 is placed on the surface 5 while being held in both hands 2 and 3, the touch sensors are turned ON and the operating button 161 is turned ON. When the blood pressure measuring device is waiting for measurement start input, the blood pressure measuring device transitions to “measurement-in-progress” mode.

Instead of the configuration in which the pulse wave sensor 20 and the electrocardiographic electrodes 30 function as touch sensors as in the third variation example, a configuration in which the operating button 161 functions as the measurement starting section and also provides a function for switching the function is also possible. In this configuration, for example, when the operating button 161 is turned from ON to OFF and then back ON (transition in sequence from the state (A) to the state (D) of FIG. 17), the blood pressure measuring device transitions from clock display mode to blood pressure measurement mode. At the same time, if the blood pressure measuring device is waiting for measurement start input, the blood pressure measuring device transition to “measurement-in-progress” mode.

In this way, the operating button 161 can serve as the switch for switching function between clock display mode and blood pressure measurement mode. Therefore, the operating button 161 can be applied as the function switching switch 42 in the above-described sixth variation example according to the first exemplary embodiment.

### Third Exemplary Embodiment

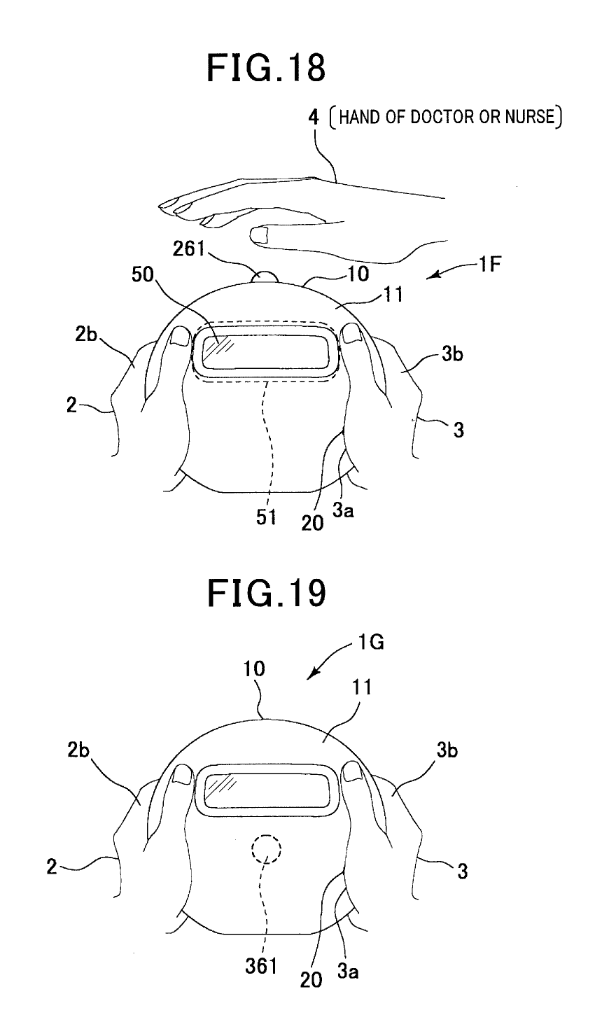

Instead of the configurations according to the first and second exemplary embodiments, a configuration is also possible in which an operating button 261 is located on the top surface of the casing 10 as shown, for example, in a blood pressure measuring device 1F in FIG. 18. In FIG. 18, illustration of the information input buttons 41 is omitted. Other configurations are the same as those according to the above-described first exemplary embodiment. Therefore, components and sections corresponding with those according to the first exemplary embodiment are given the same reference numbers. Explanations thereof are omitted.

The operating button 261 is provided in a position that is difficult to press and operate by the user himself who is holding the casing 10, but can be easily pressed and operated by a hand 4 of a third party such as a doctor or a nurse. In other words, the operating button 261 is provided in the upper portion of the casing 10 that cannot be pressed by the user himself but can be easily pressed by a third party. When the third party presses the operating button 261 while the user is holding the casing 10, the measurement circuit 60 and the calculation circuit 70 are turned ON. Acquisition of measurement information by the measurement circuit 60 is then started.

In other words, according to the third exemplary embodiment, in typical use, the third party, such as a doctor or a nurse, instructs the user to hold the casing 10 in both hands 2 and 3. After confirming that the user is holding the casing 10, the third party presses the operating button 261.

Conventional devices are not configured with the notion that a third party will press the operating button when performing measurement, but are rather configured under the premise that the user himself will perform measurement. However, as a result of the blood pressure measuring device being configured under the premise that a third party will press the operating button 261, such as that according to the third exemplary embodiment, the user can experience a sense of ease on a psychological level. In this instance, if a display section 51 displaying the measurement results is also provided on the rear surface of the casing 10, separately from the display section 50 on the front surface of the casing 10, as indicated by the broken line in FIG. 18, the third party can immediately check the measurement results.

According to the third exemplary embodiment as well, in a manner similar to that in the above-described fifth variation example according to the first exemplary embodiment, the blood pressure can be calculated without use of user information. The information input buttons 41 can be omitted. In addition, in a manner similar to that in the above-described sixth variation example according to the first exemplary embodiment, the function switching switch 42 can also be provided in the casing 10.

### Fourth Exemplary Embodiment

Instead of the operating buttons 61, 161, and 261 according to the first to third exemplary embodiments, a sensor 361, such as an acceleration sensor, that detects tilting or movement of the casing 10 may be provided within the casing 10 as shown, for example, in a blood pressure measuring device 1G in FIG. 19. In FIG. 19, illustration of the information input buttons 41 is omitted. Other configurations are the same as those according to the above-described first exemplary embodiment. Therefore, components and sections corresponding with those according to the first exemplary embodiment are given the same reference numbers. Explanations thereof are omitted.

When the user lightly shakes the casing 10 while holding the casing 10, the movement is detected by the sensor 361. The measurement circuit 60 and the calculation circuit 70 are turned ON. Acquisition of measurement information by the measurement circuit 60 is then started. In this instance, to prevent measurement from being started against the intention of the user, the blood pressure measuring device can be set such that the measurement circuit 60 and the calculation circuit 70 are turned ON when the sensor 361 detects that acceleration in a predetermined direction (such as only the vertical direction) is applied to the casing 10, or acceleration that is a predetermined reference value or greater is applied to the casing 10.

When acquisition of the measurement information is started, notification can be given to the user by this fact being displayed in the display section 50, or a buzzer sound or the like being sounded. The blood pressure measuring device may be set such that, when the user wants to stop measurement in “measurement-in-progress” mode, for example, measurement is stopped when the user again shakes the casing 10 and the sensor 361 again detects acceleration. When measurement is stopped during measurement as well, notification can be given to the user by this fact being displayed in the display section 50, or a buzzer sound or the like being sounded.

Unlike according to the first to third exemplary embodiments, according to the fourth exemplary embodiment, the operating buttons 61, 161, and 261 are not used. Therefore, the appearance of the casing 10 can be made attractive. According to the fourth exemplary embodiment as well, in a manner similar to that in the above-described fifth variation example according to the first exemplary embodiment, the blood pressure can be calculated without use of user information. The information input buttons 41 can be omitted. In addition, in a manner similar to that in the above-described sixth variation example according to the first exemplary embodiment, the function switching switch 42 can also be provided in the casing 10. In an instance in which the function switching switch 42 is provided, power can be conserved by the sensor 361 being turned ON when the blood pressure measuring device is switched from clock display mode to blood pressure measurement mode.

### Fifth Exemplary Embodiment

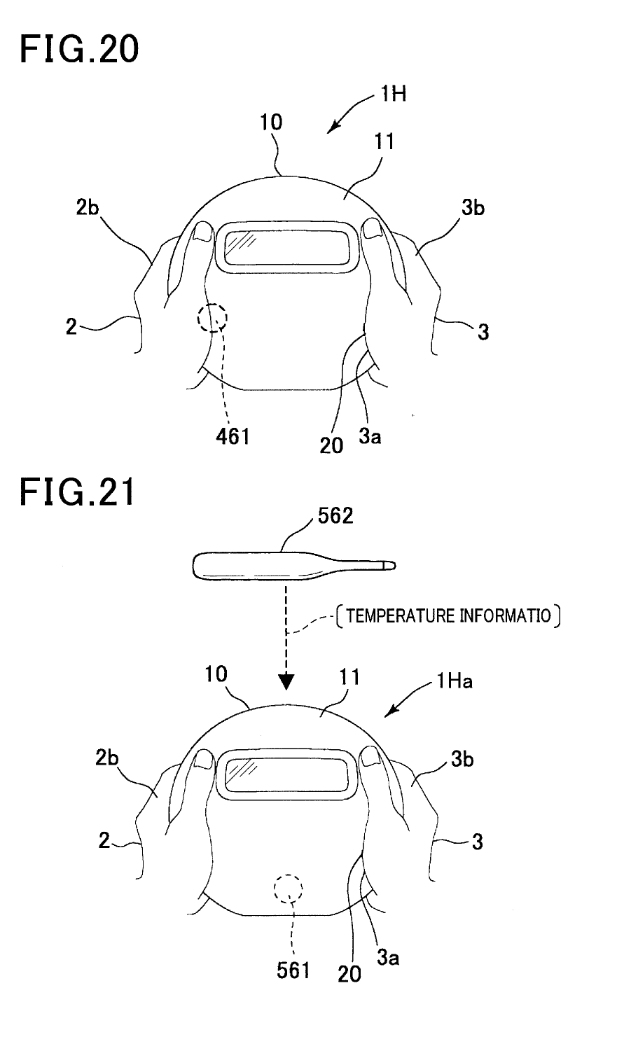

Instead of the sensor 361 such as the acceleration sensor according to the above-described fourth exemplary embodiment, a sensor 461, such as a temperature sensor, that detects temperature change on the surface of the casing 10 may be provided as shown, for example, in a blood pressure measuring device 1H in FIG. 20. In FIG. 20, illustration of the information input buttons 41 is omitted. Other configurations are the same as those according to the above-described first exemplary embodiment. Therefore, components and sections corresponding with those according to the first exemplary embodiment are given the same reference numbers. Explanations thereof are omitted.

The sensor 461 is provided in a section with which the hands 2 and 3 come into contact (including an area to which body heat is conducted). When the detected temperature exceeds a predetermined reference value, the measurement circuit 60 and the calculation circuit 70 are turned ON. Acquisition of measurement information by the measurement circuit 60 is then started. When the detected temperature falls below the reference value, acquisition of measurement information is stopped.

According to the fifth exemplary embodiment, only one sensor 461 may be provided. However, if at least a total of two sensors 461, one on each side of the casing 10 corresponding with each hand 2 and 3, are provided, detection of the casing 10 being held in the hands 2 and 3 can be made with further certainty. In this instance, the blood pressure measuring device can be set such that acquisition of measurement information starts when both temperatures detected by the sensors 461 on the left and right sides exceed the reference value. Acquisition of measurement information ends when both temperatures detected by the sensors 461 on the left and right sides fall below the reference value. When only the temperature detected by either of the sensors 461 on the left or right side falls below the reference value after the start of measurement, a judgment can be made that one of the hands has been temporarily removed from the blood pressure measuring device. Measurement can be continued.

The reference value can be set to a value lower than the standard body temperature of humans, such as 30° C. In instances in which the ambient temperature exceeds 30° C. and the sensor 461 may malfunction, the speed of change in the detected temperature may be set as the reference value. For example, a judgment can be made that the body temperature of the user is being detected when the temperature rises at a speed of 1° C. or more in a single second. Acquisition of measurement information can then be started.

Furthermore, in addition to or instead of the sensor 461, a receiving section 561 may be provided in the casing 10 that receives temperature information through a wire or wirelessly in cooperation with a clinical thermometer 562 as shown, for example, in a blood pressure measuring device 1Ha in FIG. 21 (first variation example). In FIG. 21, illustration of the information input buttons 41 is omitted. Other configurations are the same as those according to the above-described fifth exemplary embodiment. Therefore, components and sections corresponding with those according to the fifth exemplary embodiment are given the same reference numbers. Explanations thereof are omitted.

When the temperature received by the receiving section 561 exceeds a predetermined reference value, the measurement circuit 60 and the calculation circuit 70 are turned ON. Acquisition of measurement information by the measurement circuit 60 is then started. In this instance, the blood pressure measuring device 1Ha may be set such that the calculation circuit 70 displays the received temperature (the user's body temperature) together with the calculated blood pressure in the display section 50. The sensor 461 and the receiving section 561 function as a body temperature detecting section of the present invention.

According to the fifth exemplary embodiment and in the first variation example as well, in a manner similar to that in the above-described fifth variation example according to the first exemplary embodiment, the blood pressure can be calculated without use of user information. The information input buttons 41 can be omitted. In addition, in a manner similar to that in the above-described sixth variation example according to the first exemplary embodiment, the function switching switch 42 can also be provided in the casing 10. In an instance in which the function switching switch 42 is provided, power can be conserved by the sensor 461 or the receiving section 561 being turned ON when the blood pressure measuring device is switched from clock display mode to blood pressure measurement mode.

### Sixth Exemplary Embodiment

In the above-described third variation example according to the second exemplary embodiment, the pulse wave sensor 20 and the electrocardiographic electrodes 30 each also function as a touch sensor that detects contact made to the pulse wave sensor 20 or electrocardiographic electrodes 30 itself. When the pulse wave sensor 20 and the electrocardiographic electrodes 30 are both turned ON as touch sensors and the operating button 161 is turned ON, acquisition of measurement information by the measurement circuit 60 is started. However, a configuration is also possible in which the operating button 161 is omitted from the third variation example, and acquisition of measurement information by the measurement circuit 60 is started when the pulse wave sensor 20 and the electrocardiographic electrodes 30 are both turned ON as touch sensors.

According to the sixth embodiment, the pulse wave sensor 20 and the electrocardiographic electrodes 30 can function as a trigger for the measurement circuit 60 to start acquisition of measurement information, without the operating buttons 61, 161, 261, the sensors 361, 461, and the receiving section 561 being provided as the trigger. Therefore, the measurement starting section can be further simplified.

While the invention has been particularly shown and described with reference to exemplary embodiments and variation examples thereof, the invention is not limited to these embodiments and variation examples. It will be understood by those of ordinary skill in the art that various changes in form and details may be made therein without departing from the spirit and scope of the present invention as defined by the claims.

What is claimed is:

1. A blood pressure measuring device, comprising:
a casing that is held in both hands of a user;
a pair of electrocardiographic electrodes respectively provided such as to allow contact with the hands holding the casing and which detect electrocardiographic signals through the hands;
a pulse wave sensor provided such as to allow contact with either of the hands holding the casing and which detects pulse wave signals through the hand;
a measuring section that acquires measurement information including (i) a time difference between an electrocardiographic R wave based on the electrocardiographic signals and a pulse wave reference point based on the pulse wave signals, and (ii) a pulse wave amplitude based on the pulse wave signals;
a calculating section that calculates blood pressure using the measurement information;
a display section that displays the blood pressure; and
a measurement starting section configured to enable the measuring section to start acquisition of the measurement information in a state in which contact is maintained between the hands holding the casing and the corresponding electrocardiographic electrodes and pulse wave sensor.

2. The blood pressure measuring device according to claim 1, wherein:
the measurement starting section includes an operating button that is located in a position allowing contact with a palm or fingers of one hand which is not in contact with the pulse wave sensor, of the hands holding the casing.

3. The blood pressure measuring device according to claim 2, wherein:
the operating button is located in a position allowing operation by a thumb of the hand.

4. The blood pressure measuring device according to claim 3, wherein:
the casing is formed into a spherical shape; and
the operating button is located above a horizontal plane and before a vertical plane in the casing, where the horizontal plane is a horizontal plane bisecting the casing in its height direction, and the vertical plane is a vertical plane bisecting the casing in its front and rear thickness direction.

5. The blood pressure measuring device according to claim 2, wherein:
the operating button is located in a position allowing operation by a pointer finger of the hand.

6. The blood pressure measuring device according to claim 5, wherein:
the casing is formed into a spherical shape; and
the operating button is located:
above a horizontal plane and before a vertical plane in the casing, where the horizontal plane is a horizontal plane bisecting the casing in its height direction, and the vertical plane is a vertical plane bisecting the casing in its front and rear thickness direction; or
above the horizontal plane and on a side opposite to the pulse wave sensor with reference to a vertical plane in the casing, where the horizontal plane is a horizontal plane bisecting the casing in its height direction, and the vertical plane is a vertical plane bisecting the casing in its left and right direction.

7. The blood pressure measuring device according to claim 2, wherein:
the operating button is located in a position allowing operation by a palm of the hand.

8. The blood pressure measuring device according to claim 7, wherein:
the operating button is located:
below a horizontal plane and before a vertical plane in the casing, where the horizontal plane is a horizontal plane bisecting the casing in its height direction, and the vertical plane is a vertical plane bisecting the casing in its front and rear thickness direction; or
on a side opposite to the pulse wave sensor with reference to a vertical plane in the casing, where the vertical plane is a vertical plane bisecting the casing in its left and right direction.

9. The blood pressure measuring device according to claim 1, wherein:
the measurement starting section includes an operating button that is biased in a direction projecting from a bottom surface of the casing, and retracts into the casing when the casing is placed in such a manner that its bottom surface contacts a vertically lower side.

10. The blood pressure measuring device according to claim 1, wherein:
the measurement starting section includes an operating button that is provided in an upper portion of the casing in such a way as to allow the operating button to be pressed and operated by a person other than the user holding the casing.

11. The blood pressure measuring device according to claim 1, wherein:
the measurement starting section includes a sensor that detects tilting or movement of the casing.

12. The blood pressure measuring device according to claim 1, wherein:
the measurement starting section includes a body temperature detecting section that detects a body temperature of the user holding the casing.

13. The blood pressure measuring device according to claim 1, wherein:
the measurement starting section enables the measuring section to start acquisition of the measurement information, when contact between the hands holding the casing and the corresponding electrocardiographic electrodes and pulse wave sensor is detected.

14. The blood pressure measuring device according to claim 1, further comprising:
an information input section that is operated to input user information, wherein:
under a condition that the input user information is inputted through the information input section, a function of the measurement starting section is enabled.

15. A blood pressure measuring method, comprising:
detecting, by a pair of electrocardiographic electrodes respectively provided such as to allow contact with both hands of a user holding a casing, electrocardiographic signals through the hands;
detects, by a pulse wave sensor provided such as to allow contact with either of the hands holding the casing, pulse wave signals through the hand;
acquiring, by a measuring section, measurement information including (i) a time difference between an electrocardiographic R wave based on the electrocardiographic signals and a pulse wave reference point based on the pulse wave signals, and (ii) a pulse wave amplitude based on the pulse wave signals;
calculating, by a calculating section, blood pressure using the measurement information;
displaying, by a display section, the blood pressure; and
enabling, by a measurement starting section, the measuring section to start acquisition of the measurement information in a state in which contact is maintained between the hands holding the casing and the corresponding electrocardiographic electrodes and pulse wave sensor.
